(19) United States
(12) Patent Application Publication
(10) Pub. No.: US 2006/0194876 A1
(43) Pub. Date: Aug. 31, 2006

(54) Substantially pure tolterodine tartrate and process for preparing thereof

(76) Inventors: Laszlo Zsolt Kovacs, Debrecen (HU); Csaba Szabo, Debrecen (HU); Erika Magyar Molnarne, Debrecen (HU); Claude Singer, Kfar Saba (IL)

Correspondence Address: KENYON & KENYON LLP, ONE BROADWAY, NEW YORK, NY, 10004, US

(21) Appl. No.: 11/329,915

(22) Filed: Jan. 10, 2006

Related U.S. Application Data
(60) Provisional application No. 60/642,866, filed on Jan. 10, 2005.
(60) Provisional application No. 60/690,823, filed on Jun. 14, 2005.

(51) Int. Cl. A61K 31/205; C07C 59/255

(52) U.S. Cl. 514/554; 562/585

(57) ABSTRACT

The present invention provides substantially pure Tolterodine.

### RELATED APPLICATIONS

This application claims the benefit of U.S. Provisional Patent Application Nos. 60/642,866 filed Jan. 10, 2005, and 60/690,823 filed Jun. 14, 2005, the contents of which are incorporated herein by reference.

### FIELD OF THE INVENTION

The invention encompasses substantially pure Tolterodine tartrate.

### BACKGROUND OF THE INVENTION

Tolterodine is a muscarinic receptor antagonist used for the treatment of urinary urge incontinence and other symptoms of bladder overactivity. As an amine, Tolterodine forms acid addition salts when reacted with acids of sufficient strength. Pharmaceutically acceptable salts include salts of both inorganic and organic acids. The preferred pharmaceutically acceptable salt of Tolterodine is the tartrate, (R)-Tolterodine L-tartrate. The structural formula of L-(+)-tartrate of (+)-(R)-3-(2-hydroxy-5-methylphenyl)-N,N-diisopropyl-3-phenylpropylamine is shown in Formula I below.

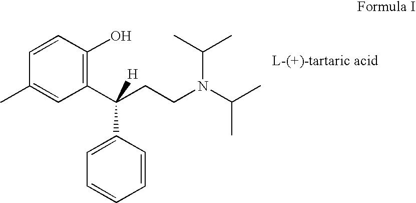

Tolterodine tartrate is marketed under the name Detrol LA® by Phannacia & Upjohn, wherein HPLC analysis of the tablets has indicated the presence of a large number of impurities, as demonstrated in Table 1. RRT is the relative retention time on HPLC.

TABLE 1

| | Related substances (area % by HPLC) | | | | | |
|---|---|---|---|---|---|---|
| | RRT | RRT | RRT | RRT | RRT | RRT |
| | 0.18 | 0.22 | 0.27 | 0.33 | 0.50 | 1.0 |
| Detrol LA (4 mg) | 0.20 | 0.16 | 0.08 | 0.11 | 0.11 | 99.27 |
| Lot: 04HPK | | | | | | |
| Expiry date: October 2004 | | | | | | |

Tolterodine tartrate and a process for its preparation were first disclosed in U.S. Pat. No. 5,382,600. The '600 patent discloses the preparation of Tolterodine by deprotecting the methylether group of the diisopropyl-[3-(2-methoxymethylphenyl)-3-phenylpropyl]-amine of formula II with boron tribromide, followed by extracting Tolterodine base of formula III with a base, and then, resolving the enantiomers with L-(+)-tartaric acid in alcohol.

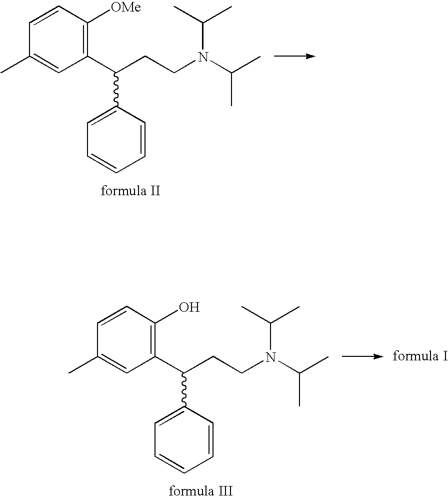

Impurities in Tolterodine or any active pharmaceutical ingredient (API) are undesirable and, in extreme cases, might even be harmful to a patient being treated with a dosage form containing the API.

In addition to stability, which is a factor in the shelf life of the API, the purity of the API produced in the commercial manufacturing process is clearly a necessary condition for commercialization. Impurities introduced during commercial manufacturing processes must be limited to very small amounts, and are preferably substantially absent. For example, the ICH Q7A guidance for API manufacturers requires that process impurities be maintained below set limits by specifying the quality of raw materials, controlling process parameters, such as temperature, pressure, time, and stoichiometric ratios, and including purification steps, such as crystallization, distillation, and liquid-liquid extraction, in the manufacturing process.

The product mixture of a chemical reaction is rarely a single compound with sufficient purity to comply with pharmaceutical standards. Side products and by-products of the reaction and adjunct reagents used in the reaction will, in most cases, also be present in the product mixture. At certain stages during processing of an API, such as Tolterodine, it must be analyzed for purity, typically, by HPLC or TLC analysis, to determine if it is suitable for continued processing and, ultimately, for use in a pharmaceutical product. The API need not be absolutely pure. Rather, purity standards are set with the intention of ensuring that an API is as free of impurities as possible, and, thus, is as safe as possible for clinical use. As discussed above, in the United States, the Food and Drug Administration guidelines recommend that the amounts of some impurities be limited to less than 0.1 percent.

Generally, side products, by-products, and adjunct reagents (collectively “impurities”) are identified spectroscopically and/or with another physical method, and then associated with a peak position, such as that in a chromatogram, or a spot on a TLC plate. (Strobel p. 953, Strobel, H. A.; Heineman, W. R., Chemical Instrumentation: A Systematic Approach, 3rd dd. (Wiley & Sons: New York 1989)). Thereafter, the impurity can be identified, e.g., by its relative position in the chromatogram, where the position in a chromatogram is conventionally measured in minutes between injection of the sample on the column and elution of the particular component through the detector. The relative position in the chromatogram is known as the “retention time.”

Thus, because of it's medical uses, it is desirable to obtain Tolterodine tartrate containing low levels of impurities.

### SUMMARY OF THE INVENTION

In one embodiment, the present invention encompasses R-Tolterodine tartrate of formula I 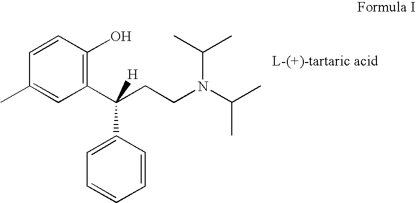
having less than about 0.5% area by HPLC of total impurities, preferably, less than about 0.3% area by HPLC of total impurities.

In another embodiment, the present invention encompasses R-Tolterodine tartrate having less than about 0.1%, and preferably, less than about 0.02% area by HPLC of at least one of impurity with a relative retention time of about 0. 18, 0.22, 0.33, or 0.50.

In yet another embodiment, the present invention encompasses an HPLC method used for the analysis of Tolterodine tartrate comprising combining an R-Tolterodine tartrate sample with a mixture of acetonitrile: water in a 1:1 ratio by volume to obtain a solution; injecting the solution into a Chromsep SS Spherisorb 3CN (100×4.6mm, 3 μm) column (or similar) maintained at a temperature of about 25° C.; gradient eluting the sample from the column at about 8 min using a mixture of acetonitrile:buffer (20:80) (referred to as eluent A) and a buffer (referred to as eluent B) as an eluent and measuring the impurity content in the relevant sample with a UV detector (preferably at a 215 nm wavelength).

In one embodiment, the present invention encompasses pharmaceutical compositions comprising Tolterodine tartrate made by the processes of the invention and at least one pharmaceutically acceptable excipient.

In another embodiment, the present invention encompasses a process for preparing a pharmaceutical formulation comprising combining the Tolterodine tartrate made by the processes of the invention, with at least one pharmaceutically acceptable excipient.

### BRIEF DESCRIPTION OF THE DRAWINGS

FIG. 1 illustrates an HPLC chromatogram of a sample of a system suitability solution.

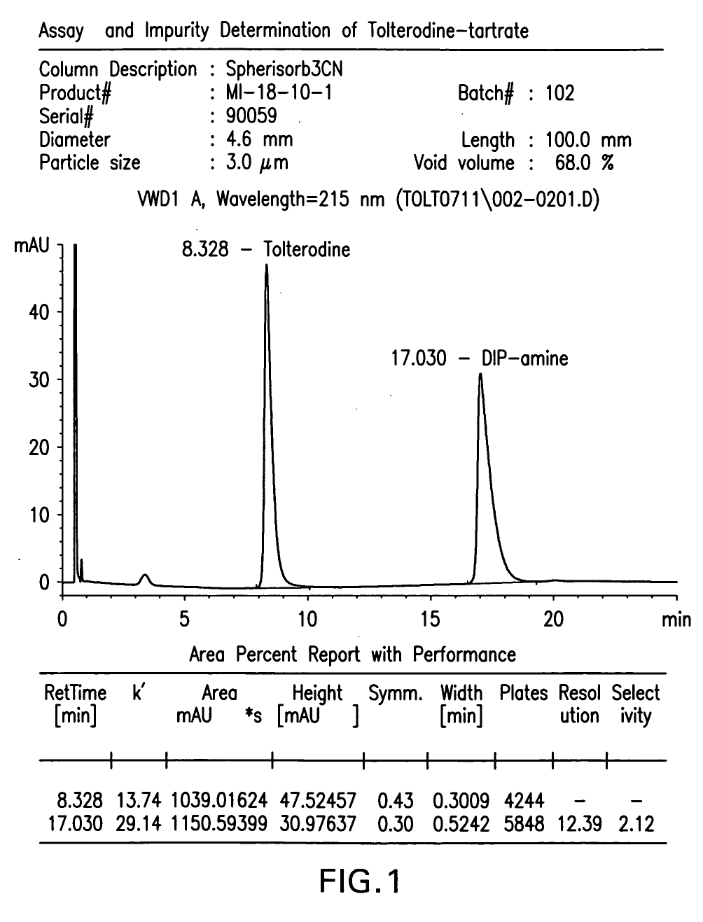

### DETAILED DESCRIPTION OF THE INVENTION

As used herein, the impurities are defined by their relative retention time (RRTs) as measured by HPLC.

The present invention encompasses R-Tolterodine tartrate of formula I

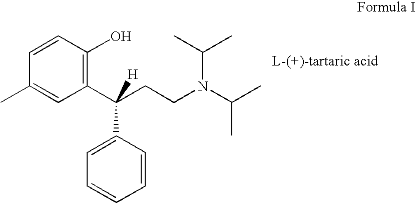

having less than about 0.5% area by HPLC of total impurities, preferably, less than about 0.3% area by HPLC of total impurities.

The present invention further encompasses R-Tolterodine tartrate having less than about 0.1%, and preferably, less than about 0.02% area by HPLC of at least one impurity with a relative retention time of about 0.18, 0.22, 0.33, or 0.50.

The present invention also encompasses an HPLC method used for the analysis of R-Tolterodine tartrate comprising combining an R-Tolterodine tartrate sample with a mixture of acetonitrile: water in a 1:1 ratio by volume, to obtain a solution; injecting the solution into a Chromsep SS Spherisorb 3CN (100×4.6mm, 3 μm) column (or similar) maintained at a temperature of about 25° C.; gradient eluting the sample from the column at about 8 min using a mixture of acetonitrile buffer (20:80) (referred to as eluent A) and a buffer (referred to as eluent B), as an eluent and measuring the impurity content in the relevant sample with a UV detector (preferably at a 215 nm wavelength).

Preferably, the buffer is an aqueous solution of KH₂PO₄, having a concentration of about 0.02 M and a pH of about 5.

The eluent used may be mixture of eluent A and eluent B, wherein the ratio of A and B varies over the time, i.e. a gradient eluent. For example, at the time 0 minutes, the eluent contains 100% of eluent A and 0% of eluent B. At 5 minutes, the eluent contains 80% of eluent A and 20% of eluent B. At 10 minutes, the eluent contains 80% of eluent A and 20% of eluent B, and at 20 minutes, the eluent contains 100% of eluent A and 0% of eluent B.

The process of the present invention for the preparation of substantially pure Tolterodine tartrate of formula I is done without requiring expensive and hazardous reagents and extensive reaction times, as compared to the product obtained by the processes of the prior art. Moreover, there is no need for isolation of Tolterodine base before resolving enantiomers to obtain the desired (R)-Tolterodine enantiomer. Hazardous reagents are avoided by using, for example, anhydrous hydrobromic acid in acetic acid, which is easier to handle, for ether cleavage. Moreover, the process of the invention prepares (R)-Tolterodine enantiomer without isolating the intermediate Tolterodine base by performing the extraction of Tolterodine base and resolution of enantiomers in the same reactor. Hence, the process of the present invention is cost effective and can be adapted to industrial scale

Tolterodine is prepared as described in U.S. Pat. No. 5,382,600, herein incorporated by reference.

R-Tolterodine tartrate of formula I of the present invention is prepared by a synthesis comprising cleaving the methyl ether of,(N,N-diisopropyl-[3-(2-methoxy-5-methylphenyl)-3-phenylpropyl]-amine) fumarate of formula II

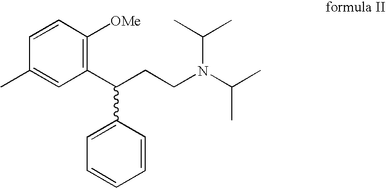

or salt thereof of formula IIa 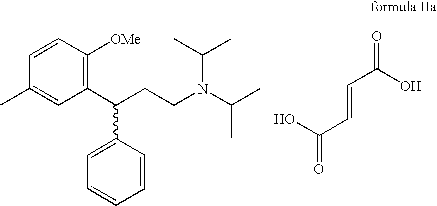
comprising treating the compound of Formula II and/or Formula IIa with a solution of hydrobromic acid in acetic acid to yield a solution, which is heated to a temperature of about 70° C. to about 120° C.; washing with a base selected from potassium hydroxide, potassium carbonate, sodium hydroxide and sodium carbonate and a solvent selected from a group consisting of water, ethylacetate, diisopropylether, toluene and mixtures thereof, to give a mixture; maintaining the mixture at a temperature of about 15° C. to about 30° C.; mixing at temperature of about room temperature to about 70° C. with a solution of L-tartaric acid in a solvent selected from ethanol and methanol; cooling to a temperature of about 5° C. to about −5° C.; recovering R-Tolterodine tartrate of formula I; and crystallizing Tolterodine tartrate of formula I.

Tolterodine HBr is prepared by cleaving the methylether group in the first step. Preferably, the concentration of the hydrobromic acid in the acetic acid solution is of about 30% to about 33%.

Preferably, the temperature is about 75° C. to about 85° C. during the first step. The first step may be carried out for about 14 hours. Preferably, the solution is stirred during the first step.

The first step leads to tolterodine HBr of the formula:

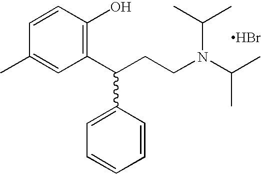

that may be recovered by cooling the solution at a temperature of about 15° C. to about 30° C., and preferably, at a temperature of about 20° C. to about 25° C. followed by addition of water, preferably, ice water, to form a slurry. The slurry is then cooled at a temperature of about 5° C. to about −5° C., while stirring for about a half an hour to about 24 hours, followed by filtration, washed with water, and drying, yielding Tolterodine HBr. Preferably, the slurry is filtered using a suction filter, and washed with ice water twice. Drying is preferably conducted at about 60° C. to about 65° C. under vacuum.

In the above-described process, preferably, the solvent is ethyl acetate. The preferred base is potassium hydroxide. Preferably, the base is added while stirring rapidly. Preferably, the mixture is maintained, at a temperature of about 20° C. to about 25° C. Preferably, the mixture is maintained for about 15 to about 30 minutes, more preferably, while stirring.

The Tolterodine hydrobromide obtained by the process of the invention may have a purity of about 98% to about 100% area by HPLC, and more preferably, of about 99% to about 100% area by HPLC.

The washing step yields Tolterodine base of formula III 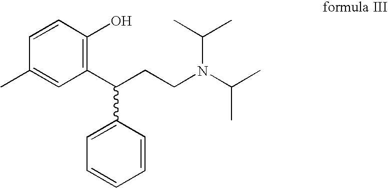
recovered by separating the organic layer and washing the organic layer with water.

R-Tolterodine tartrate of Formula I may be resolved directly from the solution of Tolterodine base of formula III obtained after the washing step.

The optical resolution step may be performed by combining a solution of Tolterodine base of formula III in a solvent selected from ethylacetate, diisopropylether and toluene with a solution of L-Tartaric acid in a solvent selected form ethanol and methanol. The preferred solvent is ethanol. The resolution is performed by adding the L-tartaric acid solution into the Tolterodine base of formula III solution, or by adding the Tolterodine base solution to the L-tartaric acid solution. The L-tartaric acid solution may be added to the solution of Tolterodine base of formula III all in one portion, meaning at one time, or over a period of time. If added over time, the addition time is preferably less than 3 hours. The reacting solutions are, preferably, combined at about room temperature.

Combining the solutions leads to a slurry, which is maintained for about 5 to about 17 hours. The slurry is maintained, preferably, at a temperature of about 5° C. to about −5° C.

Tolterodine tartrate of formula I may be recovered by filtration of the slurry, followed by washing, and drying. Preferably, the slurry is filtered using suction, washed with cold ethanol twice, and dried at 60° C. under vacuum for a period of about 3 to about 14 hours. The R-Tolterodine tartrate of formula I may be crystallized from dry ethanol.

The process may be run stepwise or concurrently, i.e., without isolation of Tolterodine base prior to the resolution step. Preferably, the process is run concurrently before the optical resolution.

Preferably, R-Tolterodine tartrate obtained by the above process contains less than about 0.5%, preferably, less than 0.3% area by HPLC of total impurities. Preferably, R-Tolterodine tartrate obtained by the above process contains less than about 0.1%, preferably, less than 0.02% area by HPLC of at least one of the impurities as measured by HPLC at relative retention times (RRTs) selected from a group consisting of about 0.18, 0.22, 0.33, and 0.50. In particular, the R-tolterodine obtained by the above process has less than about 0.1% area by HPLC of the impurity as measured by HPLC at relative retention time of about 0.18. In particular, the R-Tolterodine tartrate of the above process has less than about 0.1% area by HPLC of the impurity as measured by HPLC at relative retention time of about 0.22. In particular, the R-Tolterodine tartrate of the above process has less than about 0.1% area by HPLC of the impurity as measured by HPLC at relative retention time of about 0.33. In particular, the R-Tolterodine tartrate of the above process, has less than about 0.1% area by HPLC of the impurity as measured by HPLC at relative retention time of about 0.50.

Alternatively, the R-tolterodine obtained by the above process has less than about 0.02% area by HPLC of the impurity as measured by HPLC at relative retention time of about 0.18. In particular, the R-Tolterodine tartrate of the above process has less than about 0.02% area by HPLC of the impurity as measured by HPLC at relative retention time of about 0.22. In particular, the R-Tolterodine tartrate of the above process has less than about 0.02% area by HPLC of the impurity as measured by HPLC at relative retention time of about 0.33. In particular, the R-Tolterodine tartrate of the above process, has less than about 0.02% area by HPLC of the impurity as measured by HPLC at relative retention time of about 0.50.

The present invention comprises pharmaceutical composition comprising Tolterodine and salts thereof made by the processes of the invention, and at least one pharmaceutically acceptable excipient.

The present invention further encompasses a process for preparing a pharmaceutical formulation comprising combining the Tolterodine and salts thereof made by the processes of the invention, with at least one pharmaceutically acceptable excipient.

Methods of administration of a pharmaceutical composition of the present invention can be administered in various preparations depending on the age, sex, and symptoms of the patient. The pharmaceutical compositions can be administered, for example, as tablets, pills, powders, liquids, suspensions, emulsions, granules, capsules, suppositories, injection preparations (solutions and suspensions), and the like.

Pharmaceutical compositions of the present invention can optionally be mixed with other forms of Tolterodine tartrate and/or other active ingredients such as HMG-CoA reductase inhibitors. In addition, pharmaceutical compositions of the present invention can contain inactive ingredients such as diluents, carriers, fillers, bulking agents, binders, disintegrants, disintegration inhibitors, absorption accelerators, wetting agents, lubricants, glidants, surface active agents, flavoring agents, and the like.

Diluents increase the bulk of a solid pharmaceutical composition and can make a pharmaceutical dosage form containing the composition easier for the patient and care giver to handle. Diluents for solid compositions include, for example, microcrystalline cellulose (e.g., Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, or talc.

Carriers for use in the pharmaceutical compositions may include, but are not limited to, lactose, white sugar, sodium chloride, glucose, urea, starch, calcium carbonate, kaolin, crystalline cellulose, or silicic acid.

Binders help bind the active ingredient and other excipients together after compression. Binders for solid pharmaceutical compositions include for example acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, or starch.

Disintegrants can increase dissolution. Disintegrants include, for example, alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Disintegration inhibitors may include, but are not limited to, white sugar, stearin, coconut butter, hydrogenated oils, and the like.

Absorption accelerators may include, but are not limited to, quaternary ammonium base, sodium laurylsulfate, and the like.

Wetting agents may include, but are not limited to, glycerin, starch, and the like. Adsorbing agents used include, but are not limited to, starch, lactose, kaolin, bentonite, colloidal silicic acid, and the like.

A lubricant can be added to the composition to reduce adhesion and ease release of the product from a punch or dye during tableting. Lubricants include for example magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Glidants can be added to improve the flowability of non-compacted solid composition and improve the accuracy of dosing. Excipients that can function as glidants include for example colloidal silicon dioxide, magnesium trisilicate, powdered cellulose, starch, talc and tribasic calcium phosphate.

Flavoring agents and flavor enhancers make the dosage form more palatable to the patient. Common flavoring agents and flavor enhancers for pharmaceutical products that can be included in the composition of the present invention include for example maltol, vanillin, ethyl vanillin, menthol, citric acid, fumaric acid, ethyl maltol, and tartaric acid.

Tablets can be further coated with commonly known coating materials such as sugar coated tablets, gelatin film coated tablets, tablets coated with enteric coatings, tablets coated with films, double layered tablets, and multi-layered tablets. Capsules can be coated with shell made, for example, from gelatin and optionally contain a plasticizer such as glycerin and sorbitol, and an opacifying agent or colorant.

Solid and liquid compositions can also be dyed using any pharmaceutically acceptable colorant to improve their appearance and/or facilitate patient identification of the product and unit dosage level.

In liquid pharmaceutical compositions of the present invention, the Tolterodine tartrate forms described herein and any other solid ingredients are dissolved or suspended in a liquid carrier, such as water, vegetable oil, alcohol, polyethylene glycol, propylene glycol or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention can also contain viscosity enhancing agents to improve the mouth-feel of the product and/or coat the lining of the gastrointestinal tract. Such agents include for example acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar can be added to improve the taste. Preservatives and chelating agents such as alcohol, sodium benzoate, butylated hydroxy toluene, butylated hydroxyanisole and ethylenediamine tetraacetic acid can be added at safe levels to improve storage stability.

A liquid composition according to the present invention can also contain a buffer such as guconic acid, lactic acid, citric acid or acetic acid, sodium guconate, sodium lactate, sodium citrate or sodium acetate.

Selection of excipients and the amounts to use can be readily determined by an experienced formulation scientist in view of standard procedures and reference works known in the art.

A composition for tableting or capsule filing can be prepared by wet granulation. In wet granulation some or all of the active ingredients and excipients in powder form are blended and then further mixed in the presence of a liquid, typically water, which causes the powders to clump up into granules. The granulate is screened and/or milled, dried and then screened and/or milled to the desired particle size. The granulate can then be tableted or other excipients can be added prior to tableting, such as a glidant and/or a lubricant.

A tableting composition can be prepared conventionally by dry blending. For instance, the blended composition of the actives and excipients can be compacted into a slug or a sheet and then comminuted into compacted granules. The compacted granules can be compressed subsequently into a tablet.

As an alternative to dry granulation, a blended composition can be compressed directly into a compacted dosage form using direct compression techniques. Direct compression produces a more uniform tablet without granules. Excipients that are particularly well-suited to direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention can comprise any of the aforementioned blends and granulates that were described with reference to tableting, only they are not subjected to a final tableting step.

When shaping the pharmaceutical composition into pill form, any commonly known excipient used in the art can be used. For example, carriers include, but are not limited to, lactose, starch, coconut butter, hardened vegetable oils, kaolin, talc, and the like. Binders used include, but are not limited to, gum arabic powder, tragacanth gum powder, gelatin, ethanol, and the like. Disintegrating agents used include, but are not limited to, agar, laminalia, and the like.

For the purpose of shaping the pharmaceutical composition in the form of suppositories, any commonly known excipient used in the art can be used. For example, excipients include, but are not limited to, polyethylene glycols, coconut butter, higher alcohols, esters of higher alcohols, gelatin, semisynthesized glycerides, and the like.

When preparing injectable pharmaceutical compositions, solutions and suspensions are sterilized and are preferably made isotonic to blood. Injection preparations may use carriers commonly known in the art. For example, carriers for injectable preparations include, but are not limited to, water, ethyl alcohol, propylene glycol, ethoxylated isostearyl alcohol, polyoxylated isostearyl alcohol, and fatty acid esters of polyoxyethylene sorbitan. One of ordinary skill in the art can easily determine with little or no experimentation the amount of sodium chloride, glucose, or glycerin necessary to make the injectable preparation isotonic. Additional ingredients, such as dissolving agents, buffer agents, and analgesic agents may be added. If necessary, coloring agents, preservatives, perfumes, seasoning agents, sweetening agents, and other medicines may also be added to the desired preparations during the treatment of schizophrenia.

The amount of Tolterodine tartrate or pharmaceutically acceptable salt thereof contained in a pharmaceutical composition for reducing cholesterol according to the present invention is not specifically restricted; however, the dose should be sufficient to treat, ameliorate, or reduce the condition. For example, Tolterodine tartrate may be present in an amount of about 1% to about 70%.

The dosage of a pharmaceutical composition for reducing cholesterol according to the present invention will depend on the method of use, the age, sex, weight and condition of the patient. Typically, about 1 mg to 200 mg of Tolterodine tartrate may be contained in an administration unit form, preferably a 10 mg tablet.

Having described the invention with reference to certain preferred embodiments, other embodiments will become apparent to one skilled in the art from consideration of the specification. The invention is further defined by reference to the following examples describing in detail the process and compositions of the invention. It will be apparent to those skilled in the art that many modifications, both to materials and methods, may be practiced without departing from the scope of the invention.

### EXAMPLES

The following is a general description of the parameters used to determine sample purity. Example 9 illustrates the process. To prepare the system suitability solution, a sample of 10 mg Tolterodine tartrate and 10 mg DIP Amine MDPA Fumarate (accurately weighed) was placed into a 100 ml volumetric flask. The diluent was added and after dissolving, the flask was filled with diluent (c˜100-100 μg/ml). The resolution (Rs) value was not less than 10.0 between Tolterodine and DIP Amine MDPA.

To prepare a standard stock solution for assay determination, 10 mg of Tolterodine tartrate standard was placed into a 10 ml volumetric flask. Diluent was added and after dissolution, the flask was filled with diluent (c˜1000 μg/ml). The standard solution for assay determination was prepared by diluting 1.0 ml stock solution to 10 ml with diluent (c˜100 μg/ml). The sample solution for assay determination was prepare by using a solution of ˜100 μg/ml with diluent from all types of samples.

To determine the impurity content, a standard stock solution for impurity content determination was prepared by accurately weighing 10 mg Tolterodine tartrate standard into a 10 ml volumetric flask, dissolving in diluent, and subsequently filling the flask with diluent (c˜1000 μg/ml). The standard solution for impurity content determination was prepared by diluting 1.0 ml stock solution to 20 ml with diluent (c˜50 μg/ml) and then diluting 1.0 ml of the solution to 100 ml with diluent (c˜0.5 μg/ml). The sample solution for impurity content determination was prepared using a sample solution of −500 μg/ml with diluent from all types of samples.

The blank was diluent only.

The calculation for assay and impurity content was performed as follows:

$$Content ( \% ) = \frac{{A}_{sample} * {C}_{std} * {P}_{std}}{{A}_{std} * {C}_{sample}}$$

Where Aₛₐₘₚₗₑ is the area of the peak of required component in the chromatogram of the sample solution. A_{std} is the average area of the Tolterodine peak in the chromatograms of the standard solution. Cₛₐₘₚₗₑ is the concentration of the sample solution (μg/ml). C_{std} is the concentration of Tolterodine in the standard solution (μg/ml). P_{std} is the purity of the standard (%).

### Example 1: Preparation of Tolterodine Hydrobromide

A solution was formed by combining the fumarate salt of N,N-diisopropyl-[3-(2-methoxy-5-methylphenyl)-3-phenylpropyl]-amine of structural Formula II (200 g, 0.439 mol) and HBr in acetic acid (33%, 500 ml) and stirring at about 110° C. to about 115° C. for 14 hours in a glass reactor. The solution was cooled to room temperature and ice water (2000 ml) was added, forming a slurry. The slurry was cooled to 5° C.±5° C. and stirred for half an hour. The slurry was filtered using a suction filter, washed with ice water (2× with 200 ml) and dried at about 65° C. under vacuum for three days to yield Tolterodine hydrobromide (164.4 g) of 99.26% purity as determined by HPLC.

### Example 2: Preparation of Tolterodine Hydrobromide

A solution was formed by combining the fumarate salt of N,N-diisopropyl-[3-(2-methoxy-5-methylphenyl)-3-phenylpropyl]-amine of structural Formula II (50 g, 0.110 mol, HPLC purity of 99.56%) and HBr in acetic acid (33%, 125 ml) and stirring at 75° C. to 80° C. for 14 hours in a glass reactor. The solution was cooled to room temperature and ice water (2000 ml) was added, forming a slurry. The slurry was cooled to 5° C.±5° C. and stirred for half an hour. The slurry was filtered using a suction filter, washed with ice water (2× with 250 ml), and dried at 60° C. under vacuum for three days to yield Tolterodine hydrobromide (166.7 g) of 98.23% purity as determined by HPLC.

### Example 3: Preparation of Tolterodine Hydrobromide

A solution was formed by combining the fumarate salt of N,N-diisopropyl-[3-(2-methoxy-5-methylphenyl)-3-phenylpropyl]-amine of structural Formula 11 (50 g, 0.110 mol, HPLC purity of 99.67%) and HBr in acetic acid (33%, 125 ml) and stirring at 75° C. to 80° C. for 14 hours in a glass reactor. The solution was cooled to room temperature and water (500 ml) was added, forming a slurry. The slurry was cooled to 5° C.±5° C. and stirred for about 24 hours. The slurry was filtered using a suction filter, washed with ice water (2× with 40 ml), and dried at 60° C. under vacuum for about 18 hours to yield Tolterodine hydrobromide (32.1 g) of 98.90% purity as determined by HPLC.

### Example 4: Preparation of Tolterodine Tartrate

Tolterodine hydrobromide (100 g, 0.246 mol), ethyl acetate (2 L) and water (500 ml) were mixed at room temperature in a glass reactor, forming a mixture. The mixture was stirred rapidly while adding potassium hydroxide (50%, 300 ml). After stirring thoroughly for approximately 15-30 minutes, two clear homogeneous layers formed. The layers were separated, and an organic phase was obtained. The organic phase was washed with water (2× with 500 ml).

L-tartaric acid (38.33 g) dissolved in ethanol (800 ml) was added rapidly in one portion to the organic phase, at room temperature, forming a slurry. The slurry was cooled to 0° C.±5° C. over 2 hours and maintained at this temperature for about 15 hours. The slurry was filtered using a suction filter, washed with cold ethanol (2× with 100 ml), and dried at about 60° C. under vacuum for about 10 hours, yielding Tolterodine tartarate (62.6 g). The Tolterodine tartrate was recrystallized from dry ethanol twice, yielding Tolterodine tartrate (41.2 g) of 99.84% purity as determined by HPLC. Level of impurities as determined by HPLC: RRT 0.18, 0.22, 0.50: 0% area, RRT 0.33: 0.01% area.

### Example 5: Preparation of Tolterodine Tartrate

Tolterodine hydrobromide (100 g, 0.246 mol), ethyl acetate (2 L) and water (500 ml) were mixed at room temperature in a glass reactor, forming a mixture. The mixture was stirred rapidly while adding potassium hydroxide (50%, 300 ml). After stirring thoroughly for approximately 30 minutes, two clear homogeneous layers formed. The layers were separated, and an organic phase was obtained. The organic phase was washed with water (2× with 500 ml).

L-tartaric acid (38.4 g) dissolved in ethanol (800 ml) was added to the organic phase rapidly in one portion, at room temperature, forming a slurry. The slurry was cooled to 0° C.±5° C. over about 1 hour and maintained at this temperature for about 4 hours. The slurry was filtered using a suction filter, washed with cold ethanol (2× with 100 ml), and dried at about 60° C. under vacuum for about 10 hours to yield Tolterodine tartrate (65.2 g). The Tolterodine tartrate was recrystallized from dry ethanol, yielding Tolterodine tartrate (41.8 g) of 99.97% purity as determined by HPLC. Level of impurities, as determined by HPLC: RRT 0.18, 0.22, 0.33, 0.50: 0% area.

### Example 6: Preparation of Tolterodine Tartrate

Tolterodine hydrobromide (583 g, 1.434 mol), ethyl acetate (20 L) and water (5 L) were mixed at room temperature in a glass reactor, forming a mixture. The mixture was stirred rapidly while adding potassium hydroxide (50%, 1.5 L). After stirring thoroughly for approximately 30 minutes, two clear homogeneous layers formed. The layers were separated and an organic phase was obtained. The organic phase was washed with water (2× with 5 L).

L-tartaric acid (385 g) dissolved in ethanol (8 L) was added rapidly in one portion to the organic phase, at room temperature, forming a slurry. The slurry was cooled to 0° C.±5° C. over about 1 hour and maintained at this temperature for about 12 hours. The slurry was filtered using a suction filter, washed with cold ethanol (2× with 1 L), and dried at about 60° C. under vacuum for 3 hours to yield Tolterodine tartrate (310 g). The Tolterodine tartrate was recrystallized twice from dry ethanol, yielding Tolterodine tartrate (219 g) of 99.98% purity as determined by HPLC. Level of impurities as determined by HPLC: RRT 0.18, 0.22, 0.33, 0.50: 0% area.

### Example 7: Preparation of Tolterodine Tartrate

Tolterodine hydrobromide (20 g, 0.049 mol), ethyl acetate (400 ml) and water (100 ml) were mixed at room temperature in a glass reactor, forming a mixture. The mixture was stirred rapidly while adding potassium hydroxide (50%, 35 ml). After stirring thoroughly for approximately 30 minutes, two clear homogeneous layers formed. The layers were separated and an organic phase was obtained. The organic phase was washed with water (2× with 100 ml).

The organic phase was added to L-tartaric acid (7.7 g) dissolved in ethanol (160 ml) over about 30 minutes at room temperature, creating a slurry. The slurry was cooled to 0° C.±5° C. over about 2 hours and maintained at this temperature for about 4 hours. The slurry was filtered using a suction filter, washed with cold ethanol (2× with 20 ml), and dried at about 60° C. under vacuum for about 14 hours to yield Tolterodine tartrate (12.5 g). The Tolterodine tartrate (8.5 g) was recrystallized twice from dry ethanol, yielding Tolterodine tartrate (6.0 g) of 99.98% purity as determined by HPLC. Level of impurities as determined by HPLC: RRT 0.18, 0.22, 0.33, 0.50: 0% area.

### Example 8: Preparation of Tolterodine Tartrate

Tolterodine hydrobromide (20 g, 0.049 mol), ethyl acetate (400 ml) and water (100 ml) were mixed at room temperature in a glass reactor, forming a mixture. The mixture was stirred rapidly while adding potassium hydroxide (50%, 35 ml). After stirring thoroughly for approximately 30 minutes, two clear homogeneous layers formed. The layers were separated and an organic phase was obtained. The organic phase was washed with water (2× with 100 ml).

L-tartaric acid (7.7 g) dissolved in ethanol (160 ml) was added to the organic phase over about 2.5 hours at 70° C., forming a slurry. The slurry was cooled to 0° C.±5° C. over about 3 hours and maintained at this temperature for about 14 hours. The slurry was filtered using a suction filter, washed with cold ethanol (2× with 20 ml), and dried at about 60° C. under vacuum for about 3 hours to yield Tolterodine tartrate (10.8 g).

The Tolterodine tartrate (6.8 g) was recrystallized twice from dry ethanol, yielding Tolterodine tartrate (4.7 g) of 99.98% purity as determined by HPLC. Level of impurities as determined by HPLC: RRT 0.18, 0.22, 0.33, 0.50: 0% area.

### Example 9: HPLC Analysis

The purity determinations were performed using the following parameters. The column was a Chromsep SS Spherisorb 3CN (100×4.6mm, 3 μm) and the eluent comprised two mixtures. Mixture A had acetonitrile and 0.02 M KH₂PO₄ buffer (pH: 5.0) in a ratio of 20:80. Mixture B had 0.02 M KH₂PO₄ buffer (pH: 5.0). The gradient and time is illustrated in the following table:

| Time | Eluent A | Eluent B |
|---|---|---|
| [min] | [%] | [%] |
| 0.0 | 100 | 0 |
| 5.0 | 80 | 20 |
| 10.0 | 80 | 20 |
| 20.0 | 100 | 0 |

The flow rate was 2.0 ml/min, and the run time was 25 min. The column thermostat was set for 25° C. and the sample thermostat was set for 5° C. The detection wavelength was set at 215 nm. The diluent was acetonitrile: water in a 1:1 ratio by volume. The injection volume was 10 μl. The detection limit was 0.02% and the quantification limit was 0.05%. If necessary, minor modification of the flow rate was permitted.

Typical retention times and relative retention times were:

| | | |
|---|---|---|
| Tolterodine: | RT: ˜8 min | RRT: 1.00 |
| DIP Amine MDPA: | RT: ˜17 min | RRT: 2.04 |

### Example 10: Comparative Example

Commercial tablets were analyzed by HPLC and compared to samples of the present invention. Using HPLC, as defined above, the samples were analyzed to determine whether impurities were present in the sample. Table 1 summarizes the result of the comparison. RRT is the relative retention time on HPLC.

TABLE 1

| | Related substances (area % by HPLC) | | | | | |
|---|---|---|---|---|---|---|
| | RRT | RRT | RRT | RRT | RRT | RRT |
| | 0.18 | 0.22 | 0.27 | 0.33 | 0.50 | 1.0 |
| Detrol LA (4 mg) | 0.20 | 0.16 | 0.08 | 0.11 | 0.11 | 99.27 |
| (expiry date: October 2004) | | | | | | |

DL: 0.02%

Table 1 illustrates that tolterodine tartrate obtained by the process of the invention has substantially less impurities than the commercially available tolterodine sample. For example, the impurities at the relative retention times of 0.18, 0.22, and 0.50 were no longer present. Furthermore, the impurity at RRT of 0.33 was reduced from 0.11% to 0.01-0.0%, a factor of at least 10.

What is claimed is:

1. R-Tolterodine tartrate having less than about 0.5% area by HPLC of total impurities.

2. R-Tolterodine tartrate of claim 1, having less than about 0.3% area by HPLC of total impurities.

3. R-Tolterodine tartrate having less than about 0.1% area by HPLC of at least one of the impurities having relative retention times of about 0.18, 0.22, 0.33, or 0.50.

4. R-Tolterodine tartrate of claim 3, having less than about 0.1% area by HPLC of the impurity as measured by HPLC at relative retention time of about 0.18.

5. R-Tolterodine tartrate of claim 3, having less than about 0.1% area by HPLC of the impurity as measured by HPLC at relative retention time of about 0.22.

6. R-Tolterodine tartrate of claim 3, having less than about 0.1% area by HPLC of the impurity as measured by HPLC at relative retention time of about 0.33.

7. R-Tolterodine tartrate of claim 3, having less than about 0.1% area by HPLC of the impurity as measured by HPLC at relative retention time of about 0.50.

8. R-Tolterodine tartrate of claim 3, having less than about 0.02% area by HPLC of at least one of the impurities with an HPLC relative retention times of about 0.18, 0.22, 0.33, and 0.50.

9. R-Tolterodine tartrate of claim 8, having less than about 0.02% area by HPLC of the impurity as measured by HPLC at relative retention time of about 0.18.

10. R-Tolterodine tartrate of claim 3, having less than about 0.02% area by HPLC of the impurity as measured by HPLC at relative retention time of about 0.22.

11. R-Tolterodine tartrate of claim 3, having less than about 0.02% area by HPLC of the impurity as measured by HPLC at relative retention time of about 0.33.

12. R-Tolterodine tartrate of claim 3, having less than about 0.02% area by HPLC of the impurity as measured by HPLC at relative retention time of about 0.50.

13. An HPLC method comprising the steps of:
(a) combining an R-Tolterodine tartrate sample with a mixture of acetonitrile: water in a 1:1 ratio by volume, to obtain a solution;
(b) injecting the solution into a Chromsep SS Spherisorb 3CN (100×4.6mm, 3 μm) column (or similar) maintained at a temperature of about 25° C.;
(c) gradient eluting the sample from the column at about 8 min using a mixture of acetonitrile:buffer (20:80) (referred to as eluent A) and a buffer (referred to as eluent B) as an eluent; and
(d) measuring the impurity content in the relevant sample with a UV detector (at a 215 nm wavelength).

14. The process of claim 13, wherein the buffer is an aqueous solution of KH₂PO₄, having a concentration of about 0.02 M and a pH of about 5.

15. The process of claim 13, wherein the ratio of eluent A and eluent B varies over the time.

16. The process of claim 15, wherein the ratio of eluent A and eluent B at the time 0 minutes, is 100% of eluent A and 0% of eluent B.

17. The process of claim 15 wherein the ratio of eluent A and eluent B at 5 minutes and 10 minutes, is 80% of eluent A and 20% of eluent B.

18. The process of claim 15, wherein the ratio of eluent A and eluent B at the time 20 minutes, is 100% of eluent A and 0% of eluent B.

20. Pharmaceutical composition comprising the Tolterodine Tartrate of any one of claims 1 to 18, and at least one pharmaceutically acceptable excipient.

21. A process for preparing a pharmaceutical formulation comprising combining the Tolterodine tartrate of any one of claims 1 to 18, with at least one pharmaceutically acceptable excipient.
